(19) United States
(12) Patent Application Publication
(10) Pub. No.: US 2003/0199781 A1
(43) Pub. Date: Oct. 23, 2003

(54) Automatic electroencephalogram analysis apparatus and method

(75) Inventors: Yukihiro Tsuboshita, Kanagawa (JP); Isao Yamaguchi, Kanagawa (JP); Kazuhisa Ichikawa, Kanagawa (JP)

Correspondence Address: OLIFF & BERRIDGE, PLC, P.O. BOX 19928, ALEXANDRIA, VA, 22320, US

(73) Assignee: FUJI XEROX CO., LTD., Tokyo (JP)

(21) Appl. No.: 10/418,209

(22) Filed: Apr. 18, 2003

(30) Foreign Application Priority Data
Apr. 22, 2002 (JP) 2002-119057

(51) Int. Cl. A61B 5/04

(52) U.S. Cl. 600/544

(57) ABSTRACT

Discrimination-target electroencephalographic data input from a discrimination-target electroencephalographic data input portion is converted into feature parameters on a phase space and feature parameters on a frequency space by a feature parameter extracting portion. By use of feature parameters generated likewise from a reference learning electroencephalographic data set input from a reference learning electroencephalographic data set input portion, a reference data space calculating portion calculates a mean, a variance, and an inverse matrix of a correlation matrix of the reference learning electroencephalographic data set. These are used as a reference data space. A Mahalanobis distance calculating portion obtains a Mahalanobis distance from the mean, the variance, and the inverse matrix of the correlation matrix of the reference learning electroencephalographic data set calculated as a reference data space, and the feature parameters calculated from the discrimination-target electroencephalographic data. A judgment portion judges normality/abnormality of the discrimination-target electroencephalogram according to the Mahalanobis distance.

### The present disclosure relates to the subject matter contained in Japanese Patent Application No. 2002-119057 filed on Apr. 22, 2002, which is incorporated herein by reference in its entirety.

### BACKGROUND OF THE INVENTION

1. Field of the Invention

The present invention relates to an automatic electroencephalogram analysis technique for automatically diagnosing psychoneurotic disease such as schizophrenia, manic-depressive or epilepsy by use of electroencephalographic data.

2. Description of the Related Art

Electroencephalogram diagnosis in the related art is based on visual judgment of time-series electroencephalographic data by a skilled medical doctor. Thus, there is a problem that the judgment differs from one doctor to another due to their subjectivity, or the work cannot be turned over by any other staff than skilled medical doctors.

In addition, for example, as for electroencephalographic data handled for diagnosis of a patient contracting epilepsy, data gathered for 24 hours has to be analyzed because it cannot be seen when the patient will have a fit. It is therefore necessary to make a diagnosis on a mass of data manually.

### SUMMARY OF THE INVENTION

The invention is developed in consideration of the foregoing problems and an object of the invention is to provide an automatic electroencephalogram analysis technique in which normality/abnormality of an electroencephalogram can be grasped quantitatively so that those other than skilled medical doctors can make an objective judgment in a simple and easy way. It is another object of the invention to provide an automatic electroencephalogram analysis technique in which analysis of normality/abnormality of an electroencephalogram is automated so that the burden on an operating staff can be reduced.

According to an aspect of the invention, an automatic electroencephalogram analysis apparatus includes an input unit, a feature parameter calculating unit, a reference data space forming unit, a separation index calculating unit, a judgment unit, and an output unit. The input unit inputs time-series electroencephalographic data. The feature parameter calculating unit calculates a feature parameter pattern having a plurality of kinds of feature parameters from the time-series electroencephalographic data. The reference data space forming unit forms a reference data space using reference learning data about the feature parameter pattern. The separation index calculating unit calculates a separation index between the feature parameter pattern calculated by the feature parameter calculating unit and the reference data space, for the time-series electroencephalographic data of a subject. The judgment unit judges existence/absence of disease including neurological disease based on the calculated separation index. The output unit outputs the existence/absence of disease of the subject based on a judgment result of the judgment unit.

### BRIEF DESCRIPTION OF THE DRAWINGS

FIG. 1 is a configuration diagram of apparatus showing an embodiment of the invention.

FIG. 2 is a diagram showing an example of an electroencephalogram plotted in time series.

FIG. 3 is a block diagram showing an example of the configuration of a feature parameter extracting portion in FIG. 1.

FIG. 4 is a block diagram showing an example of the configuration of a phase analysis portion in FIG. 3.

FIG. 5 is a diagram for explaining an electroencephalographic locus of a normal person in his/her parietal region, plotted on a phase plane V-dV/dt.

FIG. 6 is a diagram for explaining an electroencephalographic locus of an epileptic patient in his/her parietal region, plotted on the phase plane V-dV/dt.

FIG. 7 is a block diagram showing an example of the configuration of an FFT analysis portion in FIG. 3.

FIG. 8 is a diagram showing an example of a frequency spectrum of an electroencephalogram subjected to FFT conversion.

FIG. 9 is a diagram for explaining electroencephalogram measuring points by way of example.

FIG. 10 is a diagram showing comparison of Mahalanobis distances using 25 feature parameters.

FIG. 11 is a factor effect chart with respect to the 25 feature parameters.

FIG. 12 is a chart showing comparison of Mahalanobis distances when 4feature parameters calculated from FFT analysis were used.

FIG. 13 is a chart showing comparison of Mahalanobis distances when 8 feature parameters specified as prime factors in factor analysis were used.

FIG. 14 is a chart for explaining comparison of Mahalanobis distances of epileptic patients with respect to a reference space in the case of using the 25 feature parameters, in the case of using the 4 feature parameters calculated from FFT analysis and in the case of using the 8 feature parameters specified as prime factors in factor analysis.

FIG. 15 is a table showing a list of feature parameters.

FIG. 16 is a table showing indexes of used feature parameters.

FIG. 17 is a table for explaining an L32 orthogonal array.

### DETAILED DESCRIPTION OF THE PREFERRED EMBODIMENTS

In an embodiment of the invention, the existence/absence of a psychiatric disorder in an electroencephalogram is judged based on various feature parameters on a phase plane V-dV/dt and on a frequency space obtained by fast Fourier transform (FFT).

In a first method for calculating feature parameters, the feature parameters are calculated on a phase plane obtained by phase analysis performed on time-series electroencephalographic data. That is, times-series cerebral evoked potential V is plotted on the phase plane V-dV/dt so as to obtain an electroencephalographic locus. Analysis is made on the obtained electroencephalographic locus. A set of intersection points between the V-axis and the electroencephalographic locus is defined as {V₀}, and a set of intersection points between the dV/dt-axis and the electroencephalographic locus is defined as {dV/dt₀}.

Examples of feature parameters on the phase plane include an aspect ratio, a V-axis maximum value, a deviation in histograms of number of times of crossing on the V-axis (hereinafter also referred to as “V-axis skew”), a ratio of number of sub-revolutions to total number of revolutions (hereinafter also referred to as “sub/total revolution number ratio”), an RL/UB distribution ratio, an RL distribution ratio, a V-axis cross gap, and so on, each of which will be described below in detail.

In a first method for calculating the aspect ratio, the aspect ratio is calculated using a maximum value |V₀|ₘₐₓ of absolute values of values V in {V₀} and a maximum value |dV/dt₀|ₘₐₓ of absolute values of values dV/dt in {dV/dt₀}, as follows.

$$\frac{{\vert ⅆ V / ⅆ {t}_{0} \vert}_{max}}{{\vert {V}_{0} \vert}_{max}} \qquad ( 1 )$$

In a second method for calculating the aspect ratio, the aspect ratio is calculated using a mean value | V₀ₘₑₐₙ of absolute values of values V in {V₀} and a mean value |dV/dt₀|ₘₑₐₙ of absolute values of values dV/dt in {dV/dt₀}, as follows.

$$\frac{{\vert ⅆ V / ⅆ {t}_{0} \vert}_{mean}}{{\vert {V}_{0} \vert}_{mean}} \qquad ( 2 )$$

Further, in a third method for calculating the aspect ratio, the aspect ratio is calculated using a variance σ²ᵥ₀ of values V in {V₀} and a variance σ² _{dV/dt0} of values dV/dt in {dV/dt₀}, as follows.

$$\frac{{σ}_{dV / {dt}_{0}}^{2}}{{σ}_{{V}_{0}}^{2}} \qquad ( 3 )$$

The V-axis maximum value is a maximum value of absolute values of values V in {V₀}, that is, the following value.

|V_{0|}ₘₐₓ (4)

The method for calculating the deviation in distribution of histograms of number of times of crossing on the v-axis (V-axis skew) is expressed using a normal distribution N(x) obtained using histograms H(x) of {V₀}, the mean V₀ₘₑₐₙ and the variance σ²_{VO} of values V in {V₀}, as follows.

$${\sum }_{x \geq 0}^{} \frac{H ( x ) - N ( x )}{N ( 0 )} - {\sum }_{x < 0}^{} \frac{H ( x ) - N ( x )}{N ( 0 )} \qquad ( 5 )$$

The method for calculating the ratio of the number of sub-revolutions to the total number of revolutions (sub/total revolution number ratio) will be described below.

The number of revolutions where the electroencephalographic locus is prevented from including the origin inside on the phase plane V-dV/dt is defined as the number of sub-revolutions N_{sub}. On the other hand, the number of revolutions regardless of whether the electroencephalographic locus includes the origin or not is defined as the total number of revolutions Nₐₗₗ. At this time, the sub/total revolution number ratio is calculated by:

$$\frac{{N}_{sub}}{{N}_{all}} \qquad ( 6 )$$

Next, the method for calculating the RL/UB distribution ratio will be described below.

The axis obtained by rotating the V-axis counterclockwise at an angle of 45° is defined as V′-axis, and the axis obtained by rotating the dV/dt-axis counterclockwise at an angle of 45° is defined as (dV/dt)′-axis. Four areas on the phase plane divided by these two axes are defined as follows.

When any point on the phase plane is expressed by (x, y),

U area: y≧x, y>−x

B area: y≦x, y<−x

R area: y<x, y≧−x

L area: y>x, y≦−x

In addition, here, sampling is carried out upon the electroencephalographic locus on the phase plane so as to regard the electroencephalographic locus as a set of points on the phase plane.

At this time, the method for calculating the RL/UB distribution ratio is expressed by:

(numberofsampledpointsinRarea)+(numberofsampledpointsinLarea)/ (numberofsampledpointsinUarea)+(numberofsampledpointsinBarea) (7)

Next, directly using of the definitions used for describing the method for calculating the RL/UB distribution ratio, the method for calculating the RL distribution ratio is expressed by:

(numberofsampledpointsinRarea)/ (numberofsampledpointsinLarea) (8)

Next, the method for calculating the V-axis cross gap will be described below.

The V-axis cross gap means the number of times with which the value of H (x) takes 0 in a section between the maximum value and the minimum value of histograms H(x) of {V₀}. This is expressed by V_{cross}.

V_{Cross} ( 9)

In a second method for calculating feature parameters in the embodiment of the invention, fast Fourier transform is applied to the time-series electroencephalographic data, and the feature parameters are calculated on a frequency space obtained thus. The feature parameters on the frequency space will be described below in detail. The feature parameters include a peak frequency, and a ratio of a peak spectrum to a second peak spectrum (hereinafter also referred to as“spectrum ratio”).

The peak frequency fₚₑₐₖ is a frequency where the spectrum has a maximum value on the frequency space.

fₚₑₐₖ ( 10)

Next, the method for calculating the ratio of the peak spectrum to the second peak spectrum (spectrum ratio) is expressed by:

$$\frac{{F}_{1}}{{F}_{2}} \qquad ( 11 )$$

where F₁, designates the maximum value of the spectrum on the frequency space, and F₂ designates the next-maximum value to the peak value F₁.

In addition, in the embodiment of the invention, the Mahalanobis-Taguchi System method (hereinafter referred to as “MTS method”) is used as the method for judging the existence/absence of psychoneurotic disease. The MTS method is a method in which with data, which is classified by human, provided as learning data, a correlation among feature parameters inherent in this learning data set is extracted so that a virtual reference data space reflecting the human ability of discrimination can be generated, and pattern recognition is performed on the basis of a Mahalanobis distance from this reference data space. Also, the method has such a feature that by giving noise to the learning data, discrimination with robustness can be attained. Furthermore, the feature parameters are optimized from the result of the discrimination so that any effective feature parameter can be extracted again. If requiring the details of the MTS method, see “Mathematical Principles of Quality Engineering” by Genichi Taguchi, Quality EngineeringVol. 6No. 6by Quality Engineering Society, pp.5-10 (1998), the entire contents of this reference incorporated herein by reference.

In the discrimination based on the MTS method, a reference data space is generated from a set of learning data, and whether unknown data belongs to the reference data space or not is judged based on its Mahalanobis distance from the generated reference data space.

The reference data space is generated in the following procedure.

[Step 1]:

Normalization of a learning data set: When the number of feature parameters of the learning data is k, the number of elements of the set of learning data is n, and value of each of learning data is xᵢⱼ (i=1, . . . , n, j=1, . . . , k) , the learning data set is converted by the following expression using the mean value mⱼ, and the variance σⱼ² Of the learning data set so as to calculate Xᵢⱼ.

$${X}_{ij} = \frac{{x}_{ij} - {m}_{j}}{\sqrt{{σ}_{j}^{2}}} ( i = 1 , ⋯ , n ; j = 1 , ⋯ , k ) \qquad ( 12 )$$

[Step 2]:

Calculation of correlation matrix: A correlation matrix R is calculated from the normalized learning data set.

$$R = [ \begin{matrix}1 & {r}_{12} & ⋯ & {r}_{1 k} \\ {r}_{21} & 1 & ⋯ & {r}_{2 k} \\ ⋮ & ⋮ & ⋰ & ⋮ \\ {r}_{k 1} & {r}_{k 2} & ⋯ & 1\end{matrix} ] \\ {r}_{ij} = \frac{1}{n} {\sum }_{l = 1}^{n} {X}_{li} {X}_{lj} ( i , j = 1 , ⋯ , k ) \qquad ( 13 )$$

[Step 3]

Calculation of inverse matrix: An inverse matrix A of the correlation matrix R is calculated.

$$A = {R}^{- 1} = [ \begin{matrix}{a}_{11} & {a}_{12} & ⋯ & {a}_{1 k} \\ {a}_{21} & {a}_{22} & ⋯ & {a}_{2 k} \\ ⋮ & ⋮ & ⋰ & ⋮ \\ {a}_{k 1} & {a}_{k 2} & ⋯ & {a}_{kk}\end{matrix} ] \qquad ( 14 )$$

The mean value mⱼ and the variance σⱼ², and the inverse matrix A of the correlation matrix R are used as a reference space pattern.

In the embodiment of the invention, the physical quantity of a scalar indicating the distance from the reference data space is defined as a separation index. In the embodiment of the invention, a Mahalanobis distance is used for calculating the separation index. The Mahalanobis distance can be regarded as “distance in consideration of correlation” among feature parameters, in comparison with a Euclidean distance used generally. In addition, the Mahalanobis distance of a subject of discrimination generally takes a value of about 3 or less when the subject of discrimination belongs to the same category of a reference data space pattern. That is, by use of the Mahalanobis distance, it can be judged whether the subject of discrimination belongs to the reference data space pattern or not.

The Mahalanobis distance of a subject of discrimination y (the number of feature parameters is k) can be calculated in the following manner.

The Mahalanobis distance D² is calculated by the following expression using a normalized value Y of the subject of discrimination y on the basis of the mean value mⱼ and the variance σⱼ² of the learning data set, which are calculated when the reference space is generated.

$$Y = \{ {Y}_{1 ,} {Y}_{2} , … , {Y}_{k} \} \\ {D}^{2} = \frac{{Y}^{T} AY}{k} \qquad ( 15 )$$

In addition, the procedure for analyzing prime factors of the respective feature parameters is defined in the MTS method. By analyzing the prime factors, feature parameters effective for discrimination can be extracted. The procedure for analyzing the prime factors is as follows.

[Step 1]:

Each feature parameter is allocated on an orthogonal array.

[Step 2]:

A reference space based on the orthogonal array is reproduced.

[Step 3: Calculation of SN ratio]:

An SN ratio is calculated based on the calculated Mahalanobis distance. The SN ratio is an index indicating the separation between the reference space and a sample to be discriminated. The increase of the SN ratio shows that data samples not belonging to the reference space can be discriminated accurately. In the embodiment of the invention, the SN ration is defined as follows.

$$η = - 10 log \frac{1}{d} ( \frac{1}{{D}_{1}^{2}} + \frac{1}{{D}_{2}^{2}} + … + \frac{1}{{D}_{d}^{2}} ) \\ η : SN ratio \\ d : number of data samples not belonging to reference space \\ used for prime factor analysis \qquad ( 16 )$$

[Step 4: Evaluation of feature parameters]:

The SN ratio when each feature parameter is used and the SN ratio when the feature parameter is not used are calculated so that a factor effect chart is created.

[Step 5: Selection of feature parameters]:

Feature parameters each providing an SN ratio reduced when it is used, that is, feature parameters each having a small factor effect are deleted on the basis of the factor effect chart.

In the embodiment of the invention, a set of feature parameters suitable for various diseases are extracted using such prime factor analysis.

Incidentally, it is also possible to perform phase analysis on an electroencephalogram to thereby extract one feature parameter such as an aspect ratio for judging disease in the electroencephalogramon the basis of the extracted feature parameter. However, in this case, usage of only one index for analyzing an electroencephalogram having a great fluctuation may lead to erroneous judgment. In addition, it is difficult to specify the threshold of the feature parameter uniquely.

Usage of a plurality of kinds of feature parameters calculated from time-series electroencephalographic data enables correct judgment. As described previously, not only the aspect ratio but also a variety of other feature parameters from phase space analysis are used, and feature parameters obtained from fast Fourier transform are used. The combination of these feature parameters and the statistical procedure performed thereon using the MTS method (multivariate analysis) open the way for automatic electroencephalogram diagnosis whose fluctuation is so great that it has been difficult to bring a judgment of normality/abnormality uniquely. By automating the analysis of electroencephalographicnormality/abnormality, the burden on an operating staff can be reduced.

Incidentally, not only can the invention be implemented as apparatus or a system, but it can be also implemented as a method. In addition, not to say, a part of the invention can be constructed as software. It goes without saying that software products used for making a computer execute such software are also included in the technical scope of the invention.

(Embodiment)

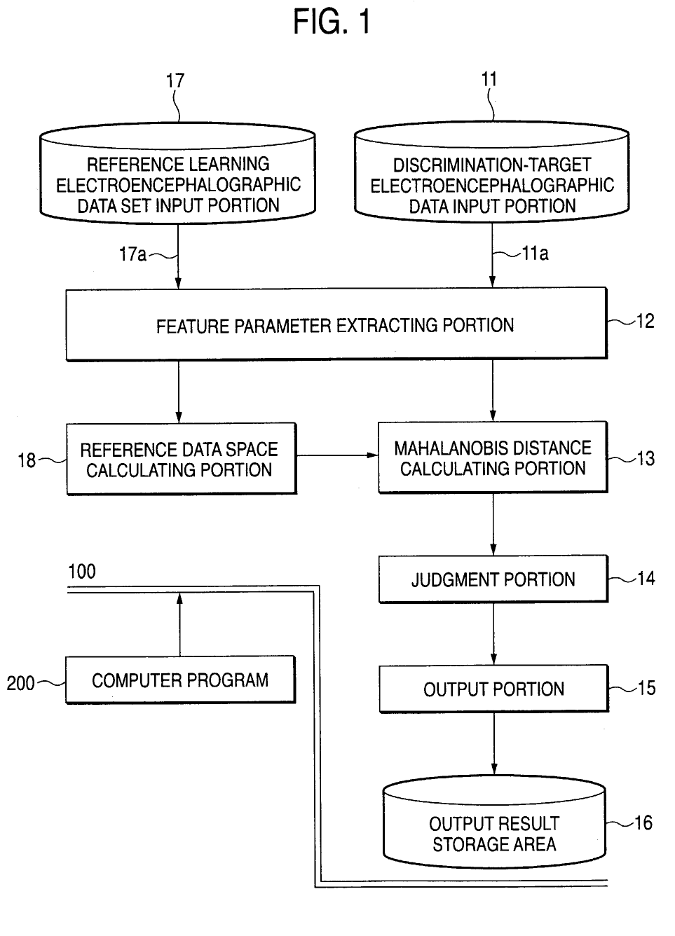

An embodiment of the invention will be described below in detail with reference to the drawings. FIG. 1 is a block diagram showing an embodiment of the invention.

In FIG. 1, an automatic electroencephalogram analyzer according to this embodiment is constituted by a discrimination-target electroencephalographic data input portion 11, a feature parameter extracting portion 12, a Mahalanobis distance calculating portionl 3 , a judgment portion 14, an output portion 15, an output result storage area 16, a reference learning electroencephalographic data set input portion 17 , a reference data space calculating portion 18, and the like. In a specific configuration, the automatic electroencephalogram analyzer can be constructed by installing a computer program 200 into a computer system 100 through a recording medium or a network. Not to say, discrete mounting can be also adopted.

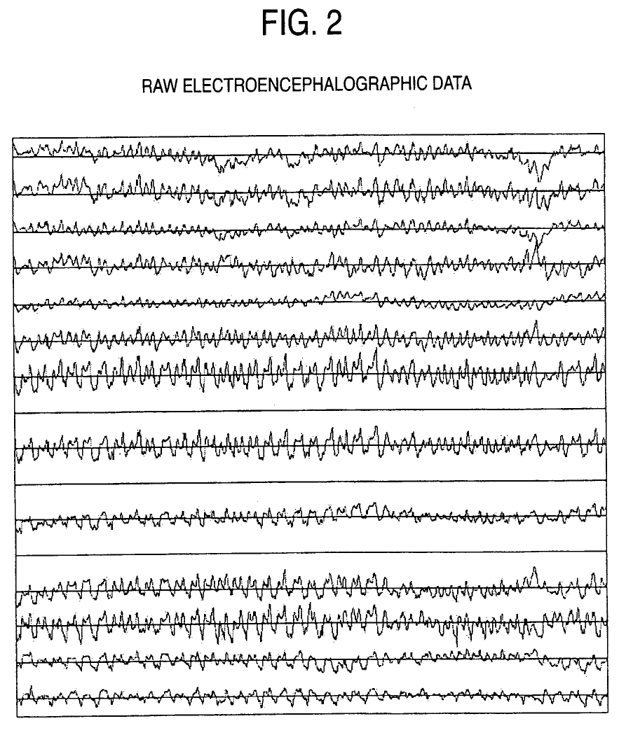

Discrimination-target electroencephalographic data 11a is input from the discrimination-target electroencephalographic data input portion 11. The discrimination-target electroencephalographic data input from the discrimination-target electroencephalographic data input portion 11 here is time-series data of cerebral evoked potential. FIG. 2 shows an electroencephalogram sampled from various portions of a head portion. The feature parameter extracting portion 12 converts the cerebral evoked potential V of the discrimination-target electroencephalographic data 11a input from the discrimination-target electroencephalographic data input portion 11 into feature parameters.

On the other hand, a reference learning electroencephalographic data set 17 a input from the reference learning electroencephalographic data set input portion 17 is converted into feature parameters by the feature parameter extracting portion 12, and then supplied to the reference data space calculating portion 18. Thus, a mean, a variance, and an inverse matrix of a correlation matrix of the reference learning electroencephalographic data set are calculated in accordance with Expressions (12) to (14). There are used as a reference data space for the following calculations.

The Mahalanobis distance calculating portion 13 obtains a Mahalanobis distance in accordance with Expression 15 from the mean, the variance, and the inverse matrix of the correlation matrix of the reference learning electroencephalographic data set calculated as a reference data space, and the feature parameters calculated from the discrimination-target electroencephalographic data 11a.

The judgment portion 14 judges normality/abnormality of the discrimination-target electroencephalogram in accordance with the Mahalanobis distance. The judgment result is stored in the output result storage area 16 by the output portion 15 .

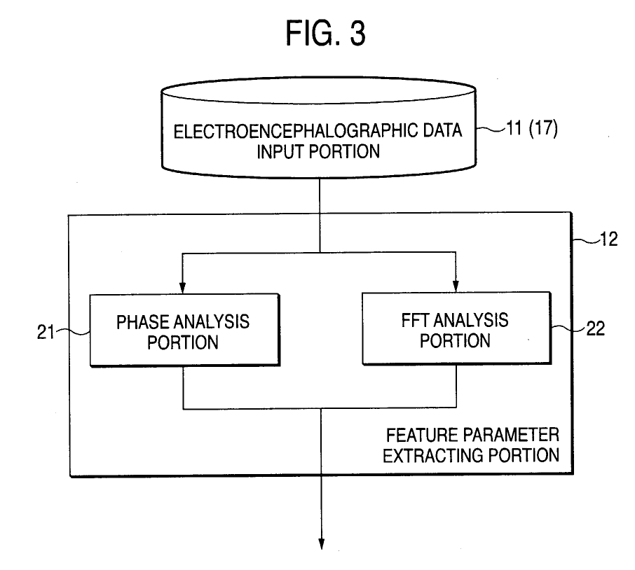

The feature parameter extracting portion 12 includes a phase analysis portion 21 for extracting phase space feature parameters and an FFT analysis portion 22 for extracting FFT feature parameters as shown in FIG. 3.

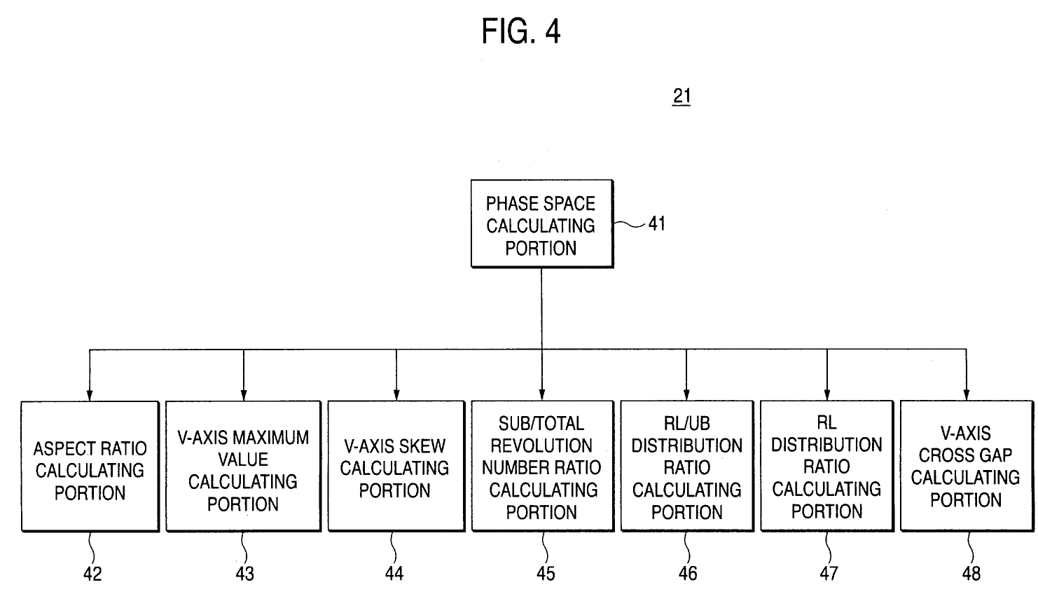

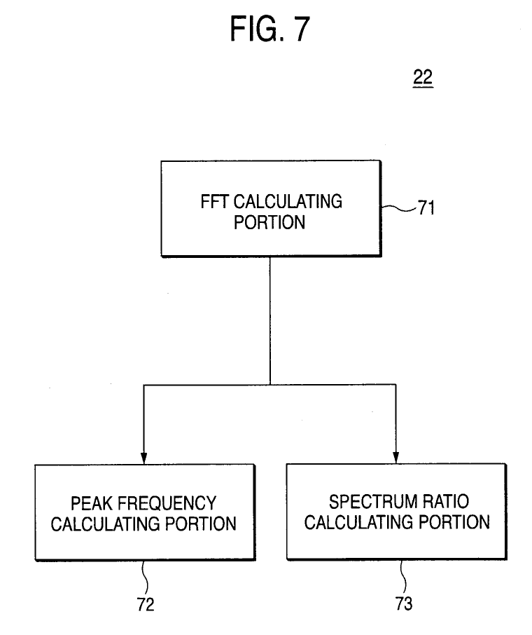

Configuration examples of the phase analysis portion 21 and the FFT analysis portion 22 are shown in FIG. 4 and FIG. 7 , respectively.

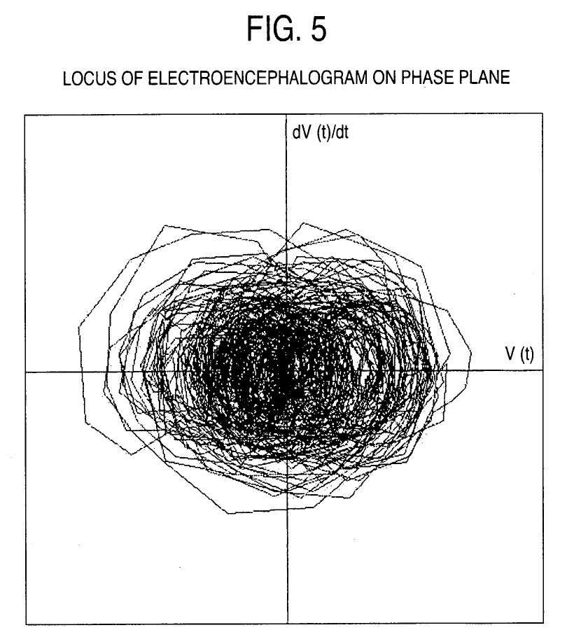

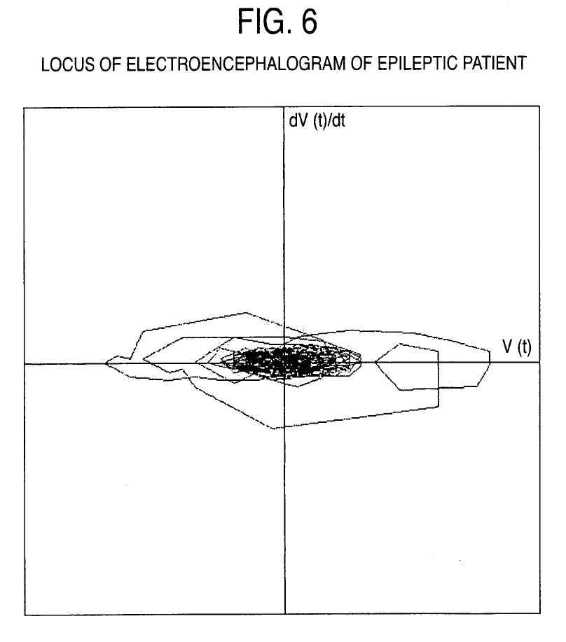

The phase analysis portion 21 shown in FIG. 4 converts the time-series electroencephalographic data into a phase space electroencephalographic locus through a phase space calculating portion 41 . Examples of time-series electroencephalographic data plotted on a phase space are shown in FIGS. 5 and 6 . FIG. 5 shows an example of a normal electroencephalographic locus, and FIG. 6 shows an example of an electroencephalographic locus having epilepsy. In FIG. 4, an aspect ratio calculating portion 42 , a V-axis maximum value calculating portion 43 , a V-axis skew calculating portion 44 , a sub/total revolution number ratio calculating portion 45 , an RL/UB distribution ratio calculating portion 46 , an RL distribution ratio calculating portion 47 and a V-axis cross gap calculating portion 48 calculate the aspect ratio, the V-axis maximum value, the V-axis skew, the sub/total revolution number ratio, the RL/UB distribution ratio, the RL distribution ratio and the V-axis cross gap in accordance with Expressions (1 ) to (9 ), respectively.

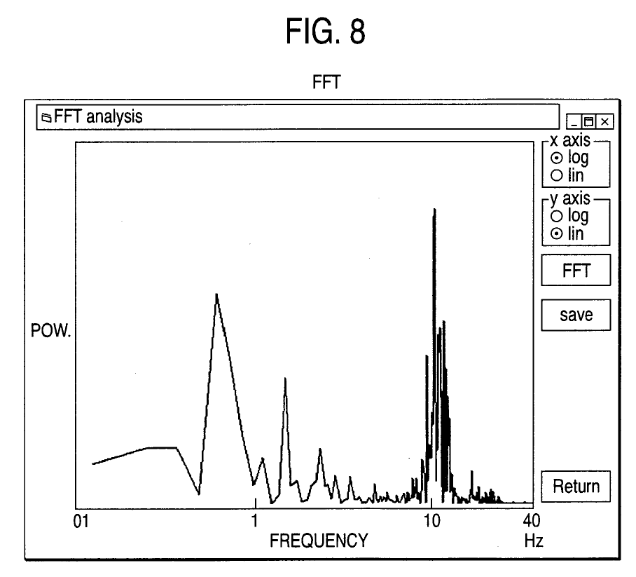

The FFT analysis portion 22 shown in FIG. 7 converts the time-series electroencephalographic data into a frequency spectrum on an FFT plane through an FFT calculating portion 71 . An example of time-series electroencephalographic data converted into a frequency spectrum is shown in FIG. 8. In FIG. 7, a peak frequency calculating portion 72 and a spectrum ratio calculating portion 73 calculates the peak frequency and the spectrum ratio in accordance with Expressions (10) and (11), respectively.

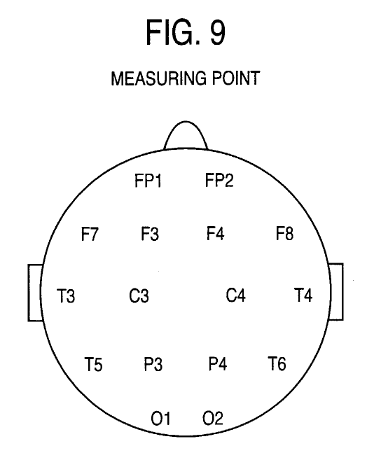

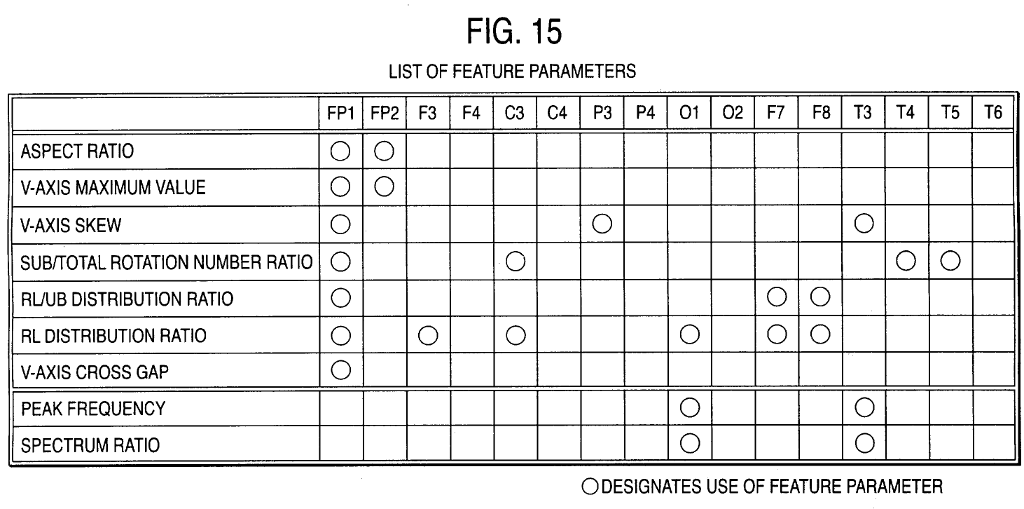

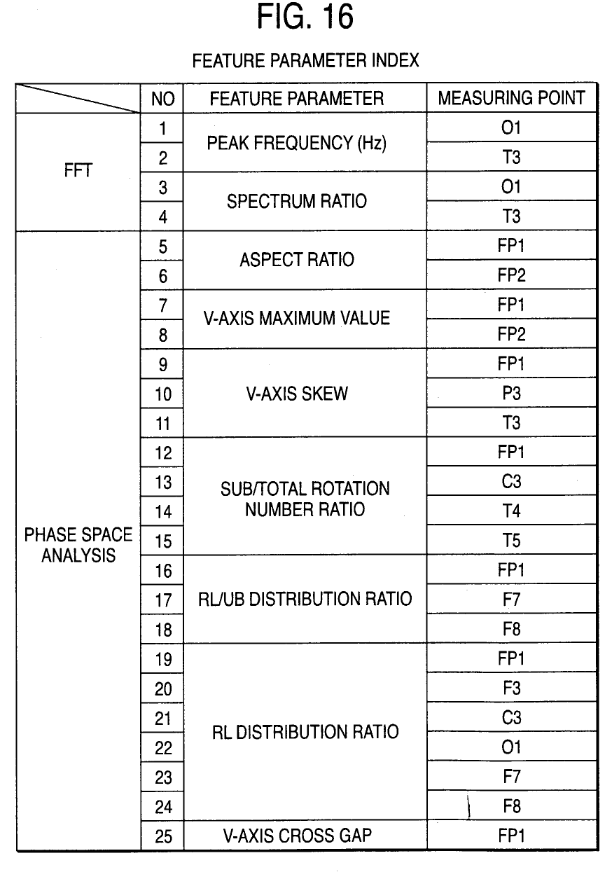

Measuring was performed upon 16 measuring points shown in FIG. 9. The number of feature parameters including the measuring points will be described. The number of categories of feature parameters was 9, and 16 measuring points were present as shown in FIG. 15. Thus, there are a total of 144 feature parameters. In this embodiment, however, verification was performed with 25 feature parameters of those feature parameters, as shown in FIG. 16.

As the reference learning electroencephalographic data set, 100 samples of normal 10-second electroencephalographic data were prepared, and a reference data space for a normal state was created based on these samples.

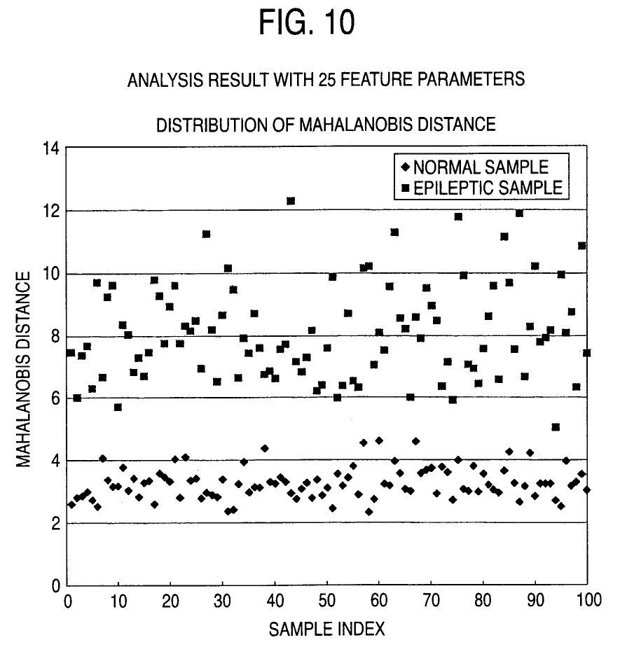

The Mahalanobis distances of 100 samples of epileptic data from the reference data space were plotted as shown in FIG. 10. It is understood that normal electroencephalographic data and epileptic electroencephalographic data are separated. However, though any Mahalanobis distance should be generally not longer than 3 when it belonged to one and the same category as the reference data space, the average of the distances of the normal samples was a comparatively large value to be 3.30 in this verification. This reason can be considered that the electroencephalograms were data having extremely great fluctuation. However, the average of the distances of the epileptic samples was 8.23, which was larger than that of the normal samples. Thus, it can be said that the normal samples and the epileptic samples are separated.

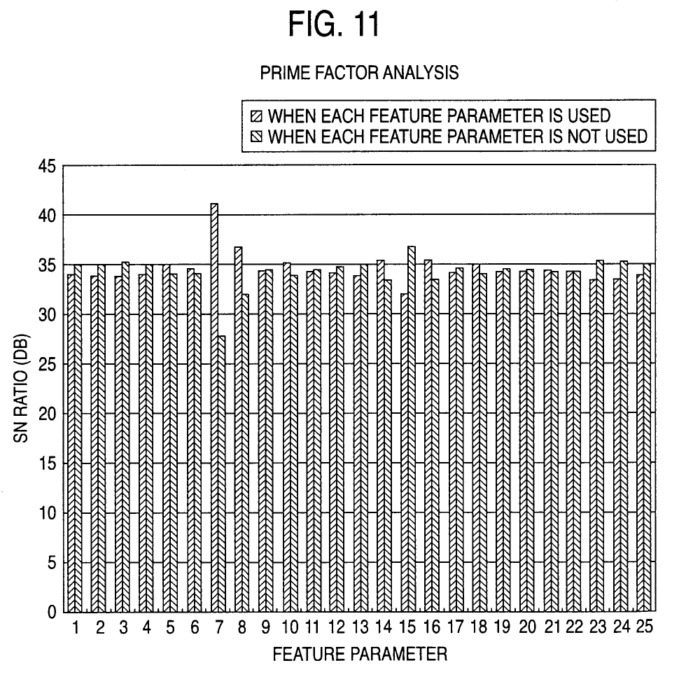

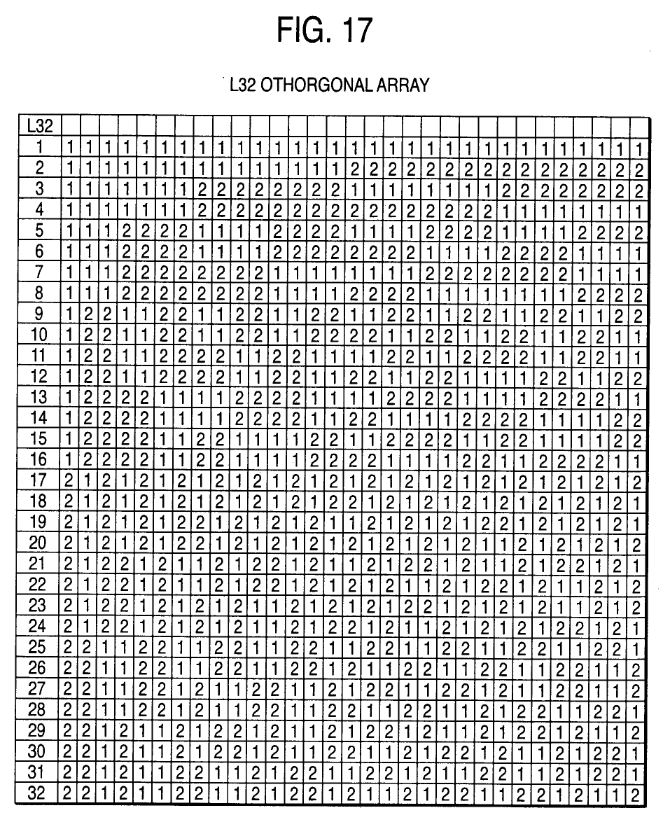

In addition, using 100 different samples of epileptic data, prime factor analysis using an L32 orthogonal array shown in FIG. 17 was performed on the 25 feature parameters selected this time. The 25 feature parameters were allocated to the columns, while “to use the feature parameter in question” was assigned to 1 in the L32 orthogonal array, and “not to use the feature parameter in question ” was assigned to 2 likewise. Then, choice/refusal of each feature parameter was made in accordance with the corresponding row in the orthogonal array. Thus, a factor effect chart was created based on the variation of the SN ratio calculated by Expression 14 . The result is shown in FIG. 11. The feature parameters 1, . . . , 25 in the abscissa of FIG. 11 correspond to the feature parameters shown in FIG. 16, respectively. According to the result, the following 8 feature parameters were specified as prime factors.

(1) aspect ratio—FP1

(2) aspect ratio—FP2

(3) V-axis maximum value—FP1

(4) V-axis maximum value—FP2

(5) V-axis skew—P3

(6) sub/total revolution number ratio—T4

(7) RL/UB distribution ratio—FP1

(8) RL/UB distribution ratio—F8

In such a manner, according to this embodiment, it can be read that the feature parameters obtained from phase analysis are greater factors for separating epilepsy and normality than the feature parameters obtained from FFT analysis. Particularly according to FIG. 11, it is understood that the greatest factor for discriminating normal electroencephalograms against epileptic electroencephalograms is the V-axis maximum value at the measuring point FP1.

Further, the Mahalanobis distances of 100 samples of epileptic data from the following three reference data spaces were compared.

(1) a reference data space using the 25 feature parameters with respect to the normal condition

(2) a reference data space reconstructed using only the 8feature parameters judged as prime factors by the prime factor analysis, with respect to the normal condition

(3) a reference data space reconstructed using only the 4 feature parameters obtained by FFT, with respect to the normal condition

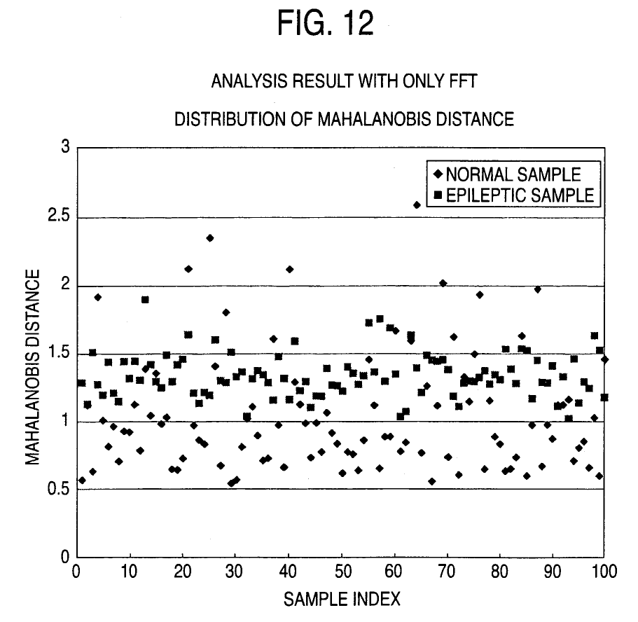

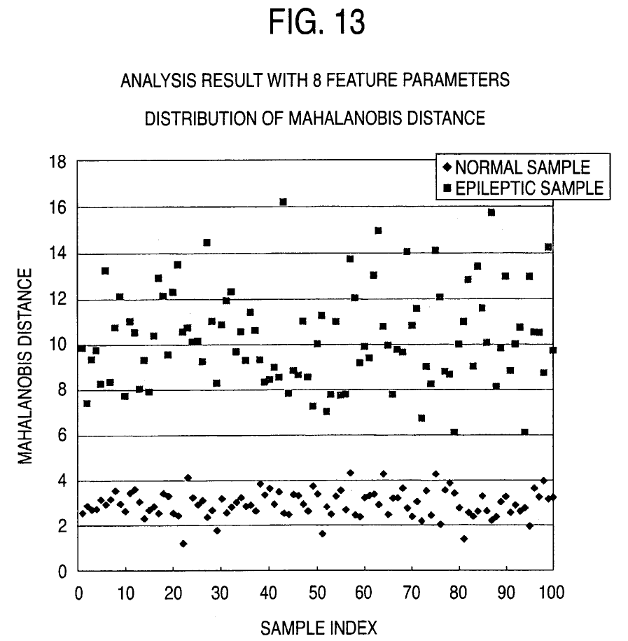

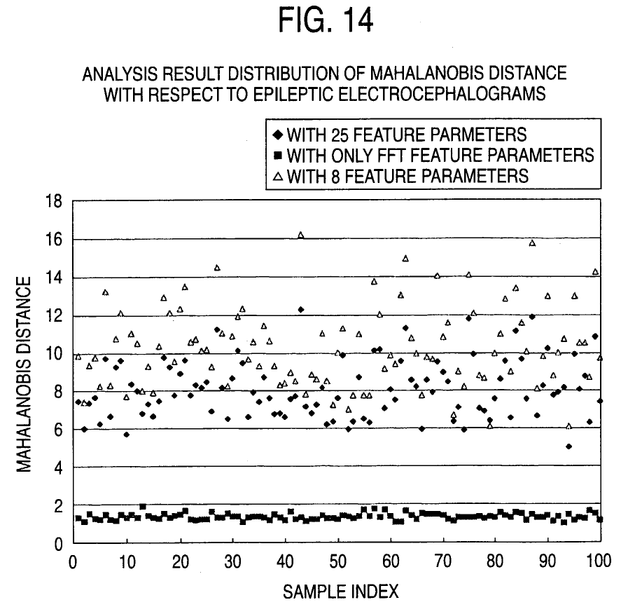

The results are shown in FIGS. 12 to 14 . Thus, it is understood that normal data and epileptic data cannot be discriminated from each other by only the 4 feature parameters obtained by FFT. Further, it can be also read that the separation between normal data and epileptic data could be made clearer when the reference data space was reconstructed with only the primary 8 feature parameters than when it was reconstructed with all the 25 feature parameters.

Incidentally, the invention is not limited to the embodiment, but various modifications can be made thereon without departing the gist of the invention. For example, although the embodiment has shown the case where a reference learning electroencephalographic data set was input from the reference learning electroencephalographic data set input portion 17 and feature parameters were extracted by the feature parameter extracting portion 12 so as to calculate a reference data space, a reference data space may be prepared in advance and held in a predetermined storage portion so as to be supplied to the Mahalanobis distance calculating portion 13 .

As is apparent from the above description, the abnormal condition which could not have been discriminated only by FFT analysis used broadly for analysis of oscillating phenomena in the related art could be discriminated correctly by use of phase space analysis. In addition, by use of a multivariate analysis method, a more robust automatic analysis technique is established. According to the inventive automatic electroencephalogram analysis method, judgment of normality/abnormality of electroencephalograms that has been made by skilled medical doctors in the related art can be performed by quantitative evaluation so that the burden on an operating staff can be reduced.

What is claimed is:

1. An automatic electroencephalogram analysis apparatus comprising:
an input unit for inputting time-series electroencephalographic data;
a feature parameter calculating unit for calculating a feature parameter pattern having a plurality of kinds of feature parameters from the time-series electroencephalographic data;
a reference data space forming unit for forming a reference data space using reference learning data about the feature parameter pattern;
a separation index calculating unit for calculating a separation index between the feature parameter pattern calculated by the feature parameter calculating unit and the reference data space, for the time-series electroencephalographic data of a subject;
a judgment unit for judging existence/absence of disease including neurological disease based on the calculated separation index; and
an output unit for outputting the existence/absence of disease of the subject based on a judgment result of the judgment unit.

2. The automatic electroencephalogram analysis apparatus according to claim 1, wherein:
the feature parameter calculating unit includes a phase analysis unit for plotting a time derivative dV/dt of cerebral evoked potential V in the time-series electroencephalographic data with respect to the cerebral evoked potential V to form an electroencephalographic locus on a phase plane V-dV/dt; and
the feature parameters are calculated on the phase plane V-dV/dt formed by the phase analysis unit.

3. The automatic electroencephalogram analysis apparatus according to claim 2, wherein the feature parameter calculating unit calculates a first histogram of intersection points between a V-axis of the phase plane V-dV/dt and the electroencephalographic locus, and a second histogram of intersection points between a dV/dt-axis of the phase plane V-dV/dt and the electroencephalographic locus.

4. The automatic electroencephalogram analysis apparatus according to claim 3, wherein the feature parameter calculating unit calculates at least one kind of aspect ratio as the feature parameters.

5. The automatic electroencephalogram analysis apparatus according to claim 4, wherein the aspect ratio is a ratio of a maximum value of absolute values of V in the first histogram to a maximum value of absolute values of dV/dt in the second histogram.

6. The automatic electroencephalogram analysis apparatus according to claim 4, wherein the aspect ratio is a ratio of a mean value of absolute values of V in the first histogram to a mean value of absolute values of dV/dt in the second histogram.

7. The automatic electroencephalogram analysis apparatus according to claim 4, wherein the aspect ratio is a ratio of a variance of V in the first histogram to a variance of dV/dt in the second histogram.

8. The automatic electroencephalogram analysis apparatus according to claim 2, wherein the feature parameter calculating unit calculates a maximum value of absolute values of V on the V-axis on a phase plane V-dV/dt as the feature parameters.

9. The automatic electroencephalogram analysis apparatus according to claim 2, wherein the feature parameter calculating unit calculates a deviation of distribution of histograms of number of times of crossing on the V-axis as the feature parameters.

10. The automatic electroencephalogram analysis apparatus according to claim 2, wherein the feature parameter calculating unit calculates a ratio of number of sub-revolutions to total number of revolutions on the phase plane V-dV/dt as the feature parameters.

11. The automatic electroencephalogram analysis apparatus according to claim 2, wherein the feature parameter calculating unit calculates an RL/UB distribution ratio on the phase plane V-dV/dt as the feature parameters.

12. The automatic electroencephalogram analysis apparatus according to claim 2, wherein the feature parameter calculating unit calculates an RL distribution ratio on the phase plane V-dV/dt as the feature parameters.

13. The automatic electroencephalogram analysis apparatus according to claim 2, wherein the feature parameter calculating unit calculates a V-axis cross gap the feature parameters.

14. Automatic electroencephalogram analysis apparatus according to claim 1, wherein:
the feature parameter calculating unit includes a fast Fourier transform analysis unit; and
the feature parameter calculating unit calculates the feature parameters on a frequency space formed by the fast Fourier transform analysis unit.

15. The automatic electroencephalogram analysis apparatus according to claim 14, wherein the feature parameter calculating unit calculates a peak frequency in the frequency space as the feature parameters.

16. The automatic electroencephalogram analysis apparatus according to claim 14, wherein the feature parameter calculating unit calculates a ratio of a peak spectrum to a second peak spectrum on the frequency space as the feature parameters.

17. The automatic electroencephalogram analysis apparatus according to claim 1, wherein a variance, a mean and an inverse matrix of a correlation matrix of the feature parameters in the reference learning data are used as the reference data space.

18. The automatic electroencephalogram analysis apparatus according to claim 1, wherein a Mahalanobis distance is used as the separation index between the feature parameters and the reference data space.

19. An automatic electroencephalogram analysis apparatus comprising:
an input unit for inputting time-series electroencephalographic data of a subject;
a feature parameter calculating unit for calculating a feature parameter pattern including a plurality of kinds of feature parameters from the time-series electroencephalographic data;
a separation index calculating unit for calculating a separation index between a reference data space formed by use of reference learning data concerning the feature parameter pattern, and the feature parameter pattern calculated for the time-series electroencephalographic data of the subject;. and
a judgment unit for judging existence/absence of disease including neurological disease based on the calculated separation index.

20. An automatic electroencephalogram analysis apparatus comprising:
an input unit for inputting time-series electroencephalographic data of a subject;
a feature parameter calculating unit for calculating feature parameters from the time-series electroencephalographic data;
a separation index calculating unit for calculating a separation index between a reference data space formed by use of reference learning data concerning the feature parameters, and the feature parameters calculated for the time-series electroencephalographic data of the subject; and
a judgment unit for judging existence/absence of disease including neurological disease based on the calculated separation index.

21. An automatic electroencephalogramanalysis method comprising:
inputting time-series electroencephalographic data of a subject;
calculating a feature parameter pattern including a plurality of kinds of feature parameters from the time-series electroencephalographic data;
calculating a separation index between a reference data space formed by use of reference learning data concerning the feature parameter pattern, and the feature parameter pattern calculated for the time-series electroencephalographic data of the subject; and
judging existence/absence of disease including neurological disease based on the calculated separation index.

22. A computer-readable recording medium recording an automatic electroencephalogram analysis computer program for making a computer execute a process comprising:
inputting time-series electroencephalographic data of a subject;
calculating a feature parameter pattern including a plurality of kinds of feature parameters from the time-series electroencephalographic data;
calculating a separation index between a reference data space formed by use of reference learning data concerning the feature parameter pattern, and the feature parameter pattern calculated for the time-series electroencephalographic data of the subject; and
judging existence/absence of disease including neurological disease based on the calculated separation index.
